(19) United States
(12) Patent Application Publication
(10) Pub. No.: US 2018/0000987 A1
(43) Pub. Date: Jan. 4, 2018

(54) POROUS BONE SUBSTITUTES AND METHOD OF PREPARING THE SAME

(71) Applicant: ELECTRONICS AND TELECOMMUNICATIONS RESEARCH INSTITUTE, Daejeon (KR)

(72) Inventors: So Young YANG, Daegu (KR); Kyu Hyung KIM, Daegu (KR); Sang Beom LEE, Daegu (KR); Soo In LEE, Daegu (KR); Eun Chang CHOI, Daegu (KR)

(73) Assignee: ELECTRONICS AND TELECOMMUNICATIONS RESEARCH INSTITUTE, Daejeon (KR)

(21) Appl. No.: 15/636,437

(22) Filed: Jun. 28, 2017

(30) Foreign Application Priority Data
Jun. 30, 2016 (KR) 10-2016-0082522

(51) Int. Cl. A61L 27/12; B33Y 70/00; B33Y 80/00; A61L 27/20; A61L 27/56; C04B 35/64; C04B 35/636; C04B 35/634; C04B 35/632; B33Y 10/00; C04B 35/447; A61L 27/50

(52) U.S. Cl. CPC A61L 27/12; C04B 35/447; B33Y 70/00; B33Y 80/00; A61L 27/20; A61L 27/50; C04B 35/64; C04B 35/6365; C04B 35/63408; C04B 35/632; B33Y 10/00; A61L 27/56; A61L 27/502; A61L 2430/02; C04B 2235/447; C04B 2235/6026; C04B 2235/606; C04B 2235/6567

(57) ABSTRACT

A method of preparing a porous bone substitute is provided. The method includes preparing a ceramic paste including calcium phosphate-based ceramics, preparing a molded article formed of the ceramic paste based on a 3D rapid prototyping method, drying the molded article, and sintering the dried molded article.

### CROSS-REFERENCE TO RELATED APPLICATION

This application claims priority to and the benefit of Korean Patent Application No. 10-2016-0082522, filed on Jun. 30, 2016, the disclosure of which is incorporated herein by reference in its entirety.

### BACKGROUND

### 1. Field of the Invention

The present invention relates to a porous bone substitute and a method of preparing the same, and more particularly, to a block-type porous bone substitute prepared using a three-dimensional (3D) rapid prototyping method and a method of preparing the same.

### 2. Discussion of Related Art

Bone substitutes used to augment bone in the bone defects caused by external injuries or surgery serve to provide a space for inducing bone regeneration, promote the fusion of a fracture site and augment a deficient alveolar bone site upon implant surgery in dental clinics. Such bone substitutes may be mainly divided into autograft, allogenic, xenograft, and alloplast (synthetic) bone substitutes.

The autograft bone substitute is the most ideal bone substitute that has the best clinical effects, but has a drawback in that it needs a second surgery to harvest bone and there is a limited bone supply and requires higher costs.

The allogenic bone substitute is a bone substitute that is prepared using bone tissues derived from cadavers or stored in tissue banks, and has advantages in that surgical sites are rapidly healed due to the absence of the second surgery, and trauma occurs less frequently, compared to the autograft bone substitute. However, when some viral diseases become pathological, the spread of diseases may be caused, and immunological rejection may occur.

The xenograft bone substitute is prepared by harvesting the bone of an animal such as cattle and subjecting the bone to chemical treatment, and thus has advantages in that it has excellent osteoconductivity and a lower risk of pathogens unlike the allogenic bone substitute, and is smoothly supplied and prepared at low costs. However, the xenograft bone substitute has a drawback in that it has a risk of transmitting a disease to animals into which it is transplanted.

Therefore, the xenograft bone substitute or the alloplast bone substitute has been recently used as the bone substitute. In the case of the alloplast bone substitute, for example, most alveolar bone substitutes have been prepared in a granule type. The granule-type bone substitutes require a surgical operation after granules are allowed to agglomerate outside using blood, etc. After the surgery, the granules should be shielded using a separate membrane such as barrier membrane so as to prevent the granules from being separated or scattered.

However, a process of suturing the membrane is a complicated surgery, and thus has a drawback in that it requires a high degree of proficiency. Also, since the bone mass, which was initially aimed to be achieved, as the granules settle down after the surgery, is not sufficiently formed, the finally formed bone may be insufficient, which leads to a larger amount of bone materials being consumed than planned.

On the other hand, the block-type bone substitute may be used to solve the drawbacks of the granule-type bone substitute such as inconvenience of surgery, difficulty in securing sufficient bone quality, etc. Also, the block-type bone substitute has advantages in that the convenience of surgery may be improved due to a decrease in the level of difficulty in surgery, and thus a surgical time may be shortened and patient satisfaction may be increased as well.

However, most block-type bone substitute products currently available on the market are the xenograft bone substitute and the allogenic bone substitute, and have a drawback in that they cannot be prepared in various shapes and sizes, in addition to the aforementioned drawbacks.

A method of preparing the block-type porous bone substitute typically includes a sponge method, direct foaming, etc. Such a block-type porous bone substitute is prepared using a suitable method selected according to the final purpose since the respective methods have their advantages and disadvantages.

The sponge method employs a principle in which polyurethane sponge is immersed into slurry and organic matter is burned so that a 3D pore structure remains in a space for the organic matter. The sponge method has an advantage in that the pore size and structure may be easily controlled according to the structure of the sponge, but has a problem in that it has a low strength and it is unsuitable for continuous mass production.

Also, the direct foaming is a method in which a bone substitute is prepared by adding various additives to slurry, foaming the resulting mixture and sintering the foamed mixture. This direct foaming method has an advantage in that the bone substitute may be easily prepared, but has a problem in that it is difficult to control the pore structure.

In this regard, Korean Patent Unexamined Publication No. 10-2013-0095014 (titled “Method Of Preparing Porous Bone Substitutes”) discloses a method of preparing porous bone substitutes using an extrusion method.

### SUMMARY OF THE INVENTION

The present invention relates to a method of preparing a porous bone substitute capable of realizing an interconnected 3D pore structure in a block type based on a 3D rapid prototyping method and controlling the granule size, porosity and pore size, and a porous bone substitute prepared using the same.

However, technical problems to be solved by this exemplary embodiment of the present invention are not limited to the technical problems as described above, and other technical problems not disclosed herein will be clearly understood from the following description.

According to an aspect of the present invention, there is provided a method of preparing a porous bone substitute, which includes preparing a ceramic paste including calcium phosphate-based ceramics, preparing a molded article formed of the ceramic paste based on a three-dimensional (3D) rapid prototyping method, drying the molded article, and sintering the dried molded article.

According to one exemplary embodiment, the preparing of the ceramic paste may further include mixing a binder including one or more selected from the group consisting of a thickening agent, a plasticizer, a lubricant, and double distilled water with the calcium phosphate-based ceramics including calcium and phosphorus.

According to one exemplary embodiment, the thickening agent may include any one selected from the group consisting of methyl cellulose, hydroxypropyl methyl cellulose, collagen, paraffin, gelatin, alginate, starch and wax, or a combination thereof.

According to one exemplary embodiment, the thickening agent may be present at a content of 1 to 20% by weight, based on the weight of the mixture including the calcium phosphate-based ceramics.

According to one exemplary embodiment, the plasticizer may include any one selected from the group consisting of polyethylene glycol, glycerol, dibutyl phthalate and dimethyl phthalate, or a combination thereof.

According to one exemplary embodiment, the plasticizer may be present at a content of 0.1 to 10% by weight, based on the weight of the mixture including the calcium phosphate-based ceramics.

According to one exemplary embodiment, the lubricant may include any one selected from the group consisting of castor oil, stearic acid, oleic acid and olive oil, or a combination thereof.

According to one exemplary embodiment, the lubricant may be present at a content of 0.1 to 10% by weight, based on the weight of the mixture including the calcium phosphate-based ceramics.

According to one exemplary embodiment, the double distilled water may be present at a content of 10 to 60% by weight, based on the weight of the mixture including the calcium phosphate-based ceramics.

According to one exemplary embodiment, the calcium phosphate-based ceramics may include any one selected from the group consisting of monocalcium phosphate monohydrate, monocalcium phosphate anhydrous, calcium metaphosphate, dicalcium phosphate dihydrate, dicalcium phosphate anhydrous, calcium pyrophosphate, octacalcium phosphate, α-tricalcium phosphate, β-tricalcium phosphate, calcium deficient hydroxyapatite, hydroxyapatite, tetracalcium phosphate and amorphous calcium phosphate, or a combination thereof.

According to one exemplary embodiment, the preparing of the molded article formed of the ceramic paste may include injecting the ceramic paste into a syringe to which an extruding machine is connected and then applying a pressure to the extruding machine based on the 3D rapid prototyping method to prepare the molded article.

According to one exemplary embodiment, the drying of the molded article may be performed at 25 to 60° C. for 12 to 48 hours.

According to one exemplary embodiment, the sintering of the dried molded article may include heating the molded article to a temperature of 1,100 to 1,200° C. at a rate of 1 to 10° C./min. and then cooling the molded article in a furnace while maintaining the molded article for 1 to 5 hours.

According to one exemplary embodiment, after the drying of the molded article, the method may further include degreasing an organic binder included in the dried molded article by heating the molded article to a temperature of 500 to 600° C. at a rate of 0.1 to 5° C./min. and then maintaining the molded article for 1 to 3 hours.

According to another aspect of the present invention, there is provided a porous bone substitute prepared using the method of preparing a porous bone substitute according to an aspect of the present invention.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a flowchart of a method of preparing a porous bone substitute according to one exemplary embodiment of the present invention;

FIGS. 2A and 2B are scanning electron microscopic (SEM) images of a powder of calcium phosphate-based ceramics;

FIGS. 3A and 3B are microscopic images of sintered bodies;

FIG. 4A is a graph obtained by performing X-ray diffraction analysis on a sintered bone substitute, and FIG. 4B and FIG. 4C is a graph obtained by measuring a compressive strength of the sintered bone substitute.

FIGS. 5A to 5D are diagrams showing microstructures of the sintered bone substitutes at respective magnifications.

### DETAILED DESCRIPTION OF EXEMPLARY EMBODIMENTS

Exemplary embodiments of the present invention will be described in detail below with reference to the accompanying drawings so that the exemplary embodiments of the present invention may be easily executed by those skilled in the prior art to which the present invention belongs. However, it should be understood that the present invention is not limited to exemplary embodiments disclosed hereinafter and is intended to be realized in various different forms. Also, in the drawings, description of parts irrelevant to the detailed description are omitted in order to describe the present invention more clearly, and like numbers refer to like elements throughout the description of the figures.

Unless the context particularly indicates otherwise through this specification, it will be further understood that the terms “comprises,” “comprising,” “includes” and/or “including,” when used herein, specify the presence of components and/or elements thereof, but do not preclude the presence or addition of other components and/or elements thereof. The term “about,” “approximately” or “substantially” used throughout this specification is intended to have meanings close to numerical values or ranges specified with an allowable error and intended to prevent accurate or absolute numerical values disclosed for understanding of the present disclosure from being illegally or unfairly used by any unscrupulous third party. The term “step of” used throughout this specification does not refer to “step for.”

The present invention relates to a method of preparing a porous bone substitute and a porous bone substitute prepared using the same.

According to one exemplary embodiment of the present invention, the biggest problems of conventional granule-type bone substitutes, such as difficulty in securing sufficient bone quality, consumption of an excessive amount of bone materials, and an increase in the level of difficulty in surgery, may be solved, and the granule size, porosity and pore size may be easily controlled using a 3D rapid prototyping method.

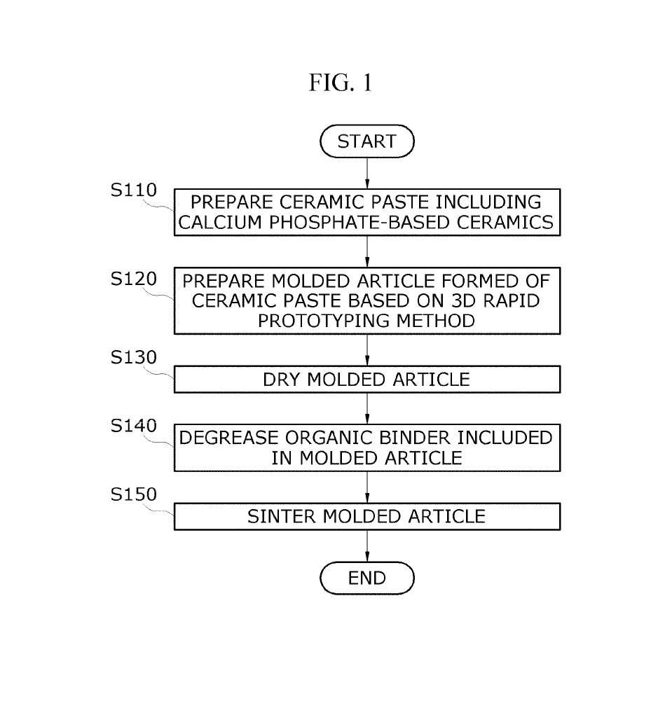

Hereinafter, a method of preparing a porous bone substitute according to one exemplary embodiment of the present invention will be described with reference to FIG. 1.

FIG. 1 is a flowchart of a method of preparing a porous bone substitute according to one exemplary embodiment of the present invention.

First of all, the method of preparing a porous bone substitute according to one exemplary embodiment of the present invention includes preparing a high-viscosity ceramic paste including calcium phosphate-based ceramics (S110).

Specifically, the preparing of the ceramic paste may include mixing a binder including one or more selected from the group consisting of a thickening agent, a plasticizer, a lubricant and double distilled water with the calcium phosphate-based ceramics including calcium and phosphorus.

The calcium phosphate-based ceramics may include any one selected from the group consisting of monocalcium phosphate monohydrate, monocalcium phosphate anhydrous, calcium metaphosphate, dicalcium phosphate dihydrate, dicalcium phosphate anhydrous, calcium pyrophosphate, octacalcium phosphate, α-tricalcium phosphate, β-tricalcium phosphate, calcium deficient hydroxyapatite, hydroxyapatite, tetracalcium phosphate and amorphous calcium phosphate, or a combination thereof.

Preferably, the calcium phosphate-based ceramics may be obtained by mixing hydroxyapatite and β-tricalcium phosphate at a ratio of 60:40 during a final sintering process.

The thickening agent may include any one selected from the group consisting of methyl cellulose, hydroxypropyl methyl cellulose, collagen, paraffin, gelatin, alginate, starch and wax, or a combination thereof.

In this case, the thickening agent may be present at a ratio of 1% by weight to 20% by weight, based on the weight of the dry mixture including the calcium phosphate-based ceramics. In this case, when the ratio of the thickening agent is less than 1% by weight, moldability may be degraded due to very low viscosity, whereas the strength after sintering may be lowered when the ratio of the thickening agent is greater than 20% by weight.

The plasticizer may include any one selected from the group consisting of polyethylene glycol, glycerol, dibutyl phthalate and dimethyl phthalate, or a combination thereof.

In this case, polyethylene glycol is preferably used as the plasticizer, but the present invention is not particularly limited thereto.

The plasticizer may be present at a ratio of 0.1% by weight to 10% by weight, based on the weight of the dry mixture including the calcium phosphate-based ceramics.

The lubricant may include any one selected from the group consisting of castor oil, stearic acid, oleic acid and olive oil, or a combination thereof.

In this case, the lubricant may be present at a ratio of 0.1% by weight to 10% by weight, based on the weight of the dry mixture including the calcium phosphate-based ceramics.

The calcium phosphate-based ceramics, and the thickening agent, the plasticizer and the lubricant, all of which are used as the binder, may be selected from the groups mentioned above, but the present invention is not particularly limited thereto.

Also, the double distilled water (2^{nd} distilled water) added during the preparing of the ceramic paste may be present at a ratio of 10% by weight to 60% by weight, based on the dry mixture including the calcium phosphate-based ceramics.

Meanwhile, the preparing of the ceramic paste as a dry mixture may be performed until a dough-like paste is formed. Also, the mixing of the ceramic paste may be performed using an alumina mortar, an agate mortar, a high-speed vortex mixer, etc. In this case, the ceramic paste may be mixed using various mixing methods without limitation to certain mixing methods

Next, a molded article formed of the ceramic paste is prepared based on a 3D rapid prototyping method (S120).

Specifically, the molded article may be prepared by injecting the ceramic paste prepared in S110 into a syringe to which an extruding machine is connected and then applying a pressure to the extruding machine based on the 3D rapid prototyping method.

In this case, the extruding machine may be used to apply a pressure to a material using a piston or screw mode. In this case, a method of applying a pressure is not limited thereto, and various methods are applicable to this method.

Also, the framework diameter of the porous bone substitute may be adjusted using nozzles having various diameters.

In addition, the size, spacing, thickness and shape of pores may be adjusted using software installed in a 3D rapid prototyping system, and the shape of the porous bone substitute may also be set to various shapes.

Then, the prototyped molded article is dried (S130).

In this case, the drying of the molded article may include drying the molded article at 25° C. to 60° C. for 12 hours to 48 hours to evaporate moisture.

Subsequently, an organic binder included in the dried molded article is degreased (S140), and the molded article is then sintered (S150).

S140 is a process of removing the organic binder included in the molded article. Here, the organic binder included in the dried molded article may be degreased by heating the molded article to a temperature of 500° C. to 600° C. at a rate of 0.1° C. to 5° C./min. and then maintaining the molded article for 1 to 3 hours. Here, when the heating temperature is less than 500° C., the organic binder may remain in the molded article. Thus, the heating temperature is preferably greater than 500° C.

In S150, the sintering is performed to improve the strength of the molded article. In this case, the sintering is performed by heating the molded article to a temperature of 1,100 to 1,200° C. at a rate of 1 to 10° C./min. and then cooling the molded article in a furnace while maintaining the molded article for 1 hour to 5 hours.

In this case, the bone substitute preferably includes hydroxyapatite and β-tricalcium phosphate at a ratio of 70 to 60:30 to 40. Here, when the heating temperature is less than 1,100° C., strength may be degraded, whereas α-tricalcium phosphate may be generated when the heating temperature is greater than 1,200° C. Therefore, the sintering is preferably performed within a temperature range of 1,100° C. to 1,200° C.

In the aforementioned description, S110 to S150 may be divided into additional steps or may be combined into fewer steps according to exemplary embodiments of the present invention. Also, some steps may be optionally omitted, and the order of the steps may also be changed.

Hereinafter, the present invention will be described in further detail with reference to preferred embodiments thereof.

1. Process of Preparing Calcium Phosphate-Based Ceramics Paste

Ethanol was added to a mixture of hydroxyapatite and β-tricalcium phosphate as starting materials, and the mixture was then ball-milled. Thereafter, the ball-milled mixture was sieved to prepare a powder having a diameter of 3 μm or less.

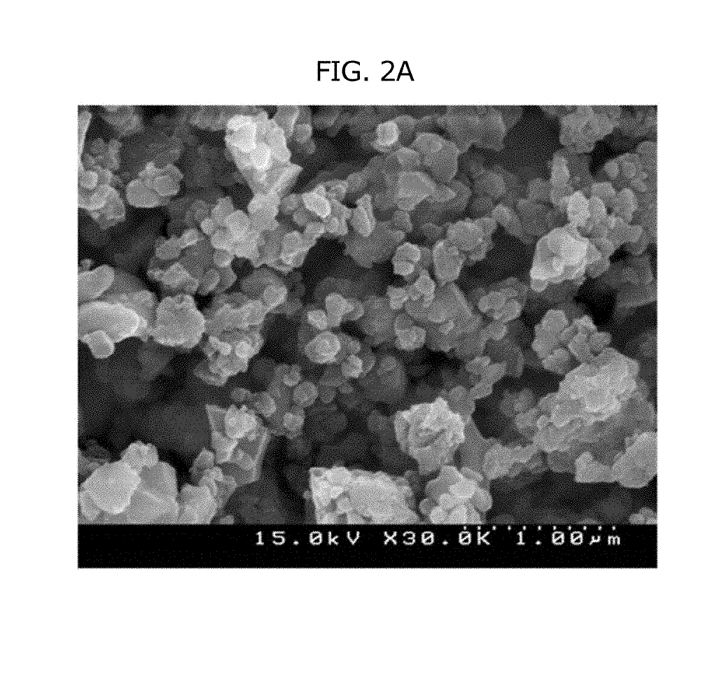

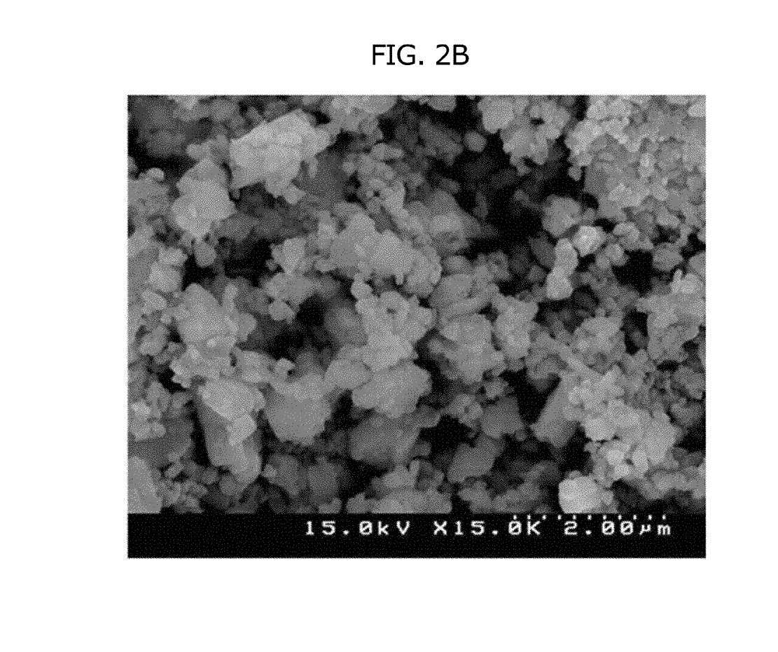

FIGS. 2A and 2B are scanning electron microscopic (SEM) images of a powder of the calcium phosphate-based ceramics.

FIGS. 2A and 2B are enlarged SEM images of sizes of 1.00 μm and 2.00 μm, respectively. As shown in FIGS. 2A and 2B, it can be seen that the powder having a nanosize of 3 μm or less was prepared for the most part.

Next, 60% by weight of double distilled water, 20% by weight of methyl cellulose as the thickening agent, 5% by weight of polyethylene glycol as the plasticizer, and 9% by weight of castor oil as the lubricant, the contents of which were based on the weight of the powder, were added to 10 g of the prepared powder, and uniformly mixed using an alumina mortar to prepare a ceramic paste.

2. Process of Preparing Bone Substitute Using 3D Rapid Prototyping Method

The ceramic paste prepared thus was injected into a syringe, and the syringe was coupled to a 3D rapid prototyping apparatus. Thereafter, the molded article was prepared.

The bone substitute was designed to be prepared in a cubic lattice model, and molded after the nozzle diameter and the interpore distance were set to 400 μm and 450 μm, respectively.

Then, the finally prepared molded article was dried at room temperature for 24 hours.

3. Process of Degreasing and Sintering Molded Article

To degrease the dried molded article, the dried molded article was heated to 500° C. at a rate of 1° C./min, and then maintained at 500° C. for 2 hours. Thereafter, the molded article was heated to 1,200° C. at a rate of 3° C./min, and then cooled in a furnace while being maintained at 1,200° C. for 3 hours, thereby completing a sintered body.

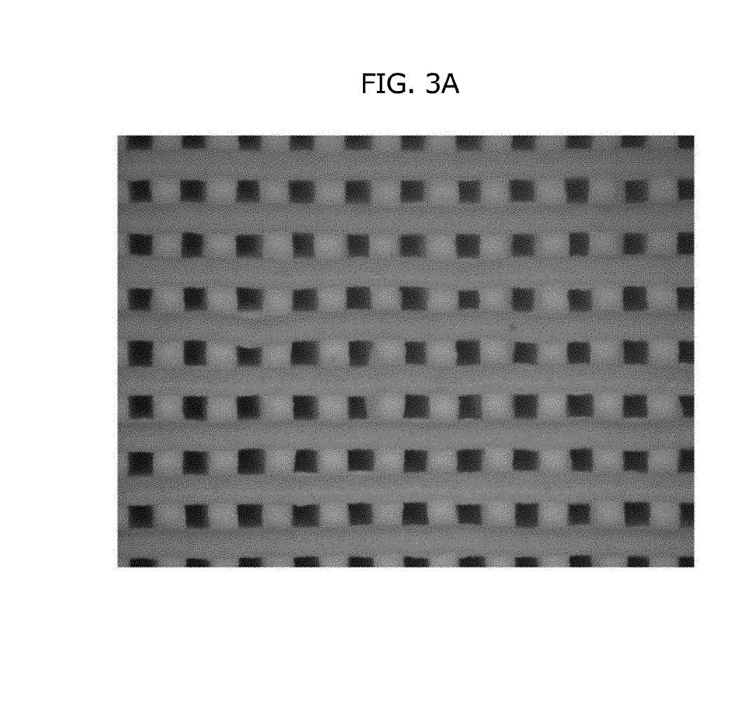

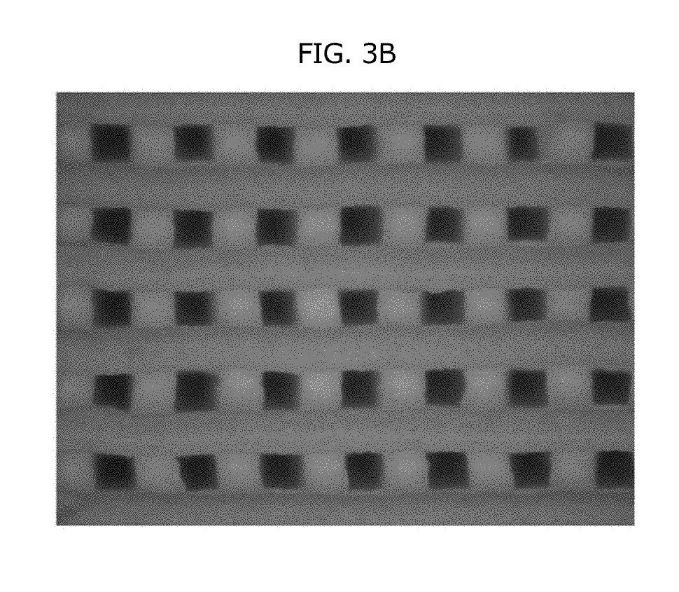

FIGS. 3A and 3B are microscopic images of the sintered bodies.

FIGS. 3A and 3B are actual microscopic images of the sintered bodies prepared according to the aforementioned process. Here, it can be seen that the molding and sintering were carried out without any cracks.

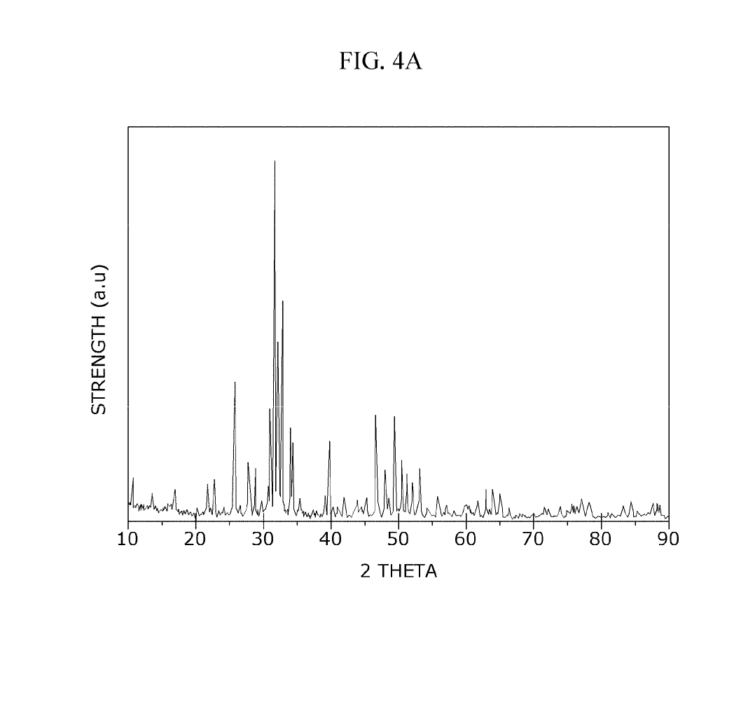

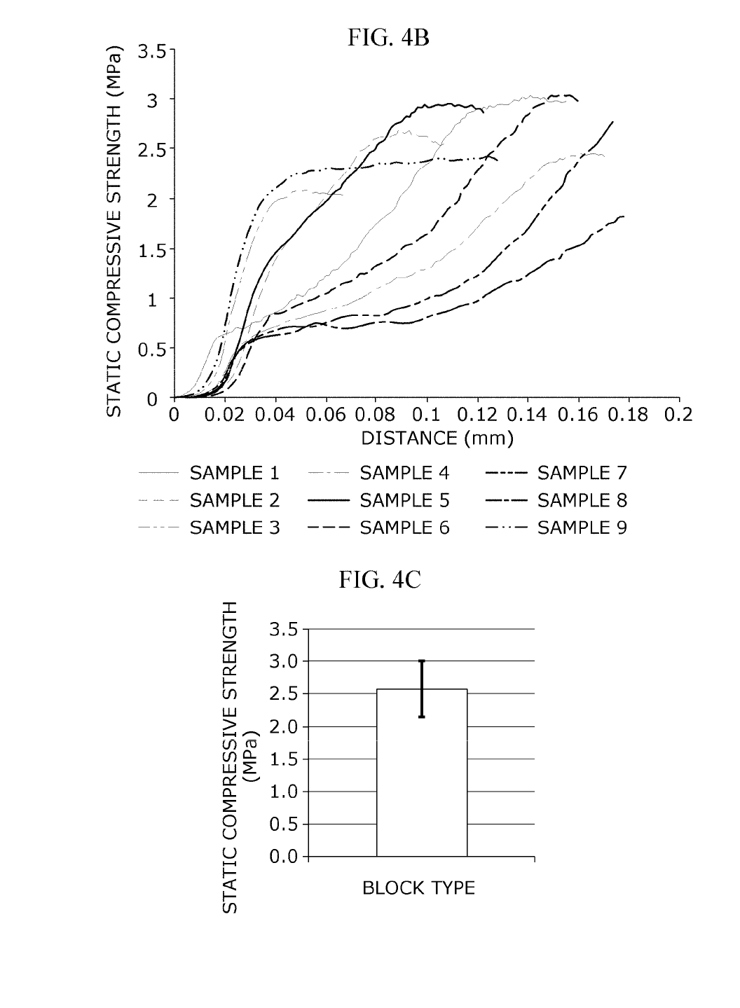

FIG. 4A is a graph obtained by performing X-ray diffraction analysis on a sintered bone substitute, and FIG. 4B and FIG. 4C is a graph obtained by measuring a compressive strength of the sintered bone substitute.

Referring to FIG. 4A, X-ray diffraction analysis was performed on the sintered bone substitute. As a result, it can be seen that the ratio of HA and β-TCP was in a range of 60 to 65:40 to 35.

Referring to FIG. 4B and FIG. 4C, it can also be seen that the sintered bone substitute had an average compressive strength of 2.52±0.4 MPa.

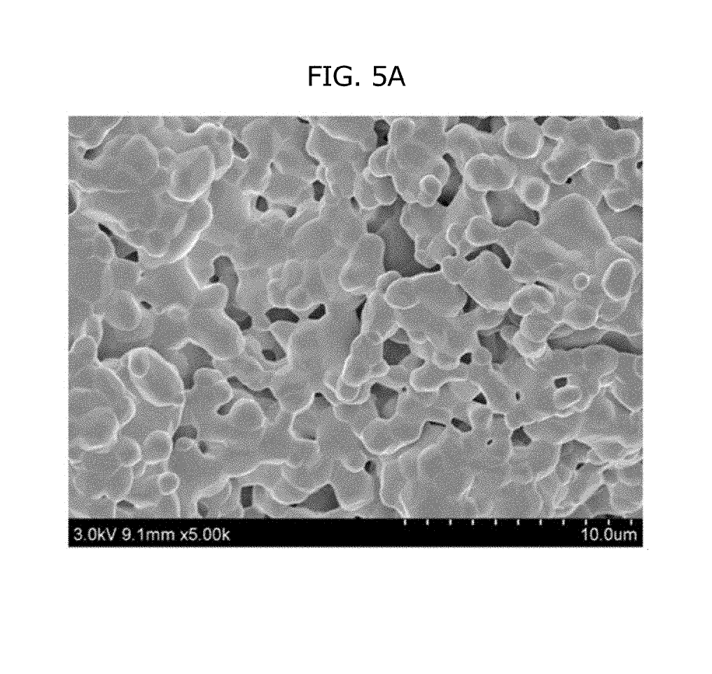

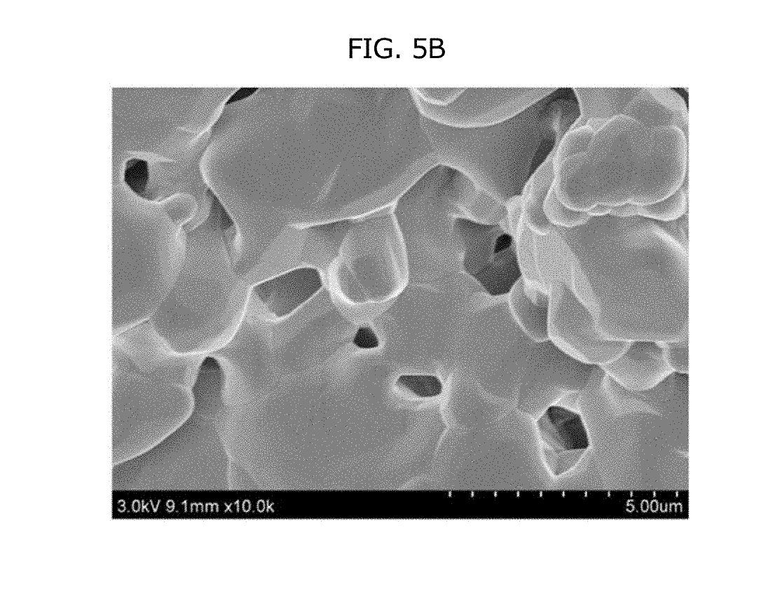

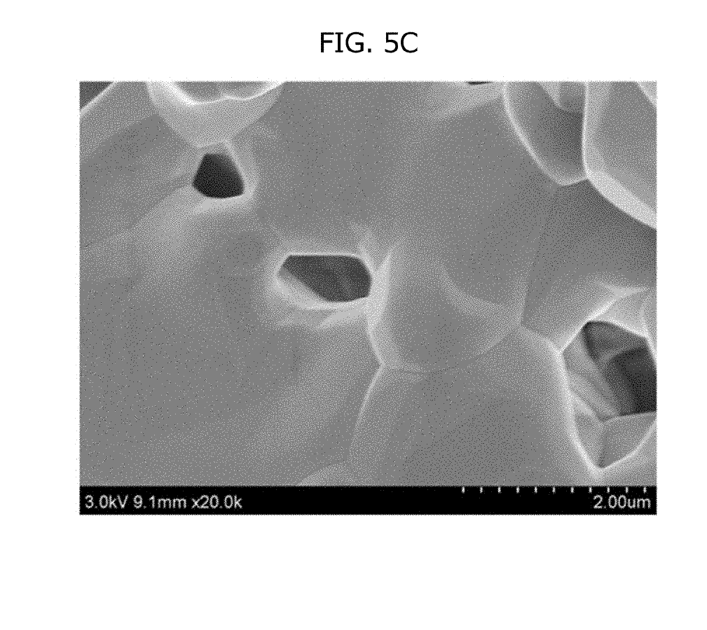

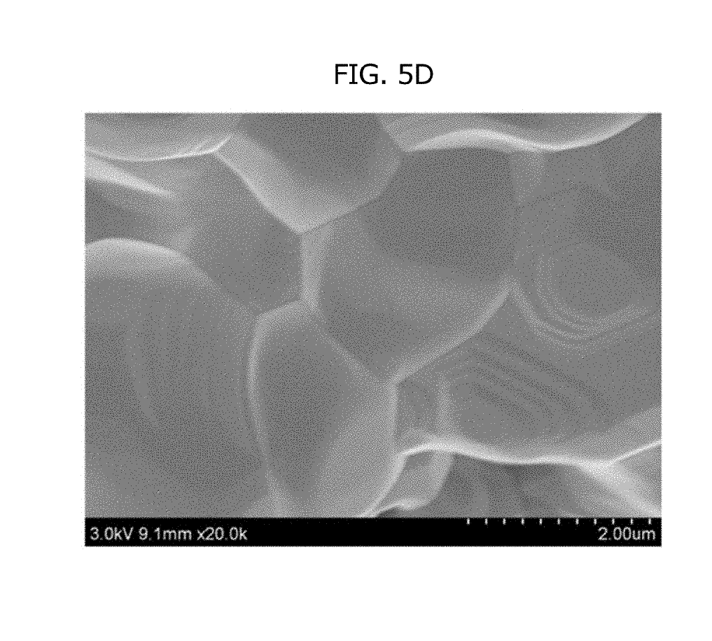

FIGS. 5A to 5D are diagrams showing microstructures of the sintered bone substitutes at respective magnifications.

FIG. 5A, FIG. 5B, and FIGS. 5C and 5D are SEM images obtained by observing the microstructures of the sintered bone substitutes at magnifications of 5,000×, 10,000×, and 20,000×, respectively. From these results, it can be seen that the sintered bone substitutes had an average porosity of 45%.

According to the aforementioned exemplary embodiment of the present invention, the bone substitutes, which have a volume most suitable for transplantation while maintaining the intact stability of raw materials, can be prepared using raw materials which have been used in the prior art.

Also, when the bone substitutes are prepared using a 3D rapid prototyping method or a 3D printer, the porous bone substitutes, which have no limitations on the sizes and shapes and are optimized for bone formation due to the ease in design of a 3D structure, can be prepared.

Further, it is possible to stack and prototype the high-viscosity ceramic paste in a 3D fashion, and also possible to control the pore size of the porous bone substitute. In particular, it can be seen that the bone substitute has a porosity of 45% or more and a mechanical strength of 2 MPa according to one exemplary embodiment of the present invention.

According to any one of the aforementioned solutions to the technical problems of the present invention, the bone substitutes, which have a volume most suitable for transplantation while maintaining the intact stability of raw materials, can be prepared using raw materials which have been used in the prior art.

Also, when the bone substitutes are prepared using a 3D rapid prototyping method or a 3D printer, the porous bone substitutes, which have no limitations on the sizes and shapes and are optimized for bone formation due to the ease in design of a 3D structure, can be prepared.

It should be understood by those skilled in the art to which the present invention pertains that the description proposed herein is given for the purpose of illustration only, and various changes and modifications can be made to the above-described exemplary embodiments of the present invention without departing from the scope of the invention. Accordingly, the exemplary embodiments of the present invention are not intended to limit the scope of the invention but to describe the invention. For example, individual components described in an integral form may be implemented in a distributed form, and individual components described in a distributed form may also be implemented in an integral form.

The scope of the present invention is defined by the appended claims, and encompasses all modifications and alterations derived from meanings, the scope and equivalents of the appended claims.

What is claimed is:

1. A method of preparing a porous bone substitute, comprising:
preparing a ceramic paste comprising calcium phosphate-based ceramics;
preparing a molded article formed of the ceramic paste based on a three-dimensional (3D) rapid prototyping method;
drying the molded article; and
sintering the dried molded article.

2. The method of claim 1, wherein the preparing of the ceramic paste further comprises:
mixing a binder comprising one or more selected from the group consisting of a thickening agent, a plasticizer, a lubricant, and double distilled water with the calcium phosphate-based ceramics comprising calcium and phosphorus.

3. The method of claim 2, wherein the thickening agent comprises any one selected from the group consisting of methyl cellulose, hydroxypropyl methyl cellulose, collagen, paraffin, gelatin, alginate, starch and wax, or a combination thereof.

4. The method of claim 3, wherein the thickening agent is present at a content of 1 to 20% by weight, based on the weight of the mixture comprising the calcium phosphate-based ceramics.

5. The method of claim 2, wherein the plasticizer comprises any one selected from the group consisting of polyethylene glycol, glycerol, dibutyl phthalate and dimethyl phthalate, or a combination thereof.

6. The method of claim 5, wherein the plasticizer is present at a content of 0.1 to 10% by weight, based on the weight of the mixture comprising the calcium phosphate-based ceramics.

7. The method of claim 2, wherein the lubricant comprises any one selected from the group consisting of castor oil, stearic acid, oleic acid and olive oil, or a combination thereof.

8. The method of claim 7, wherein the lubricant is present at a content of 0.1 to 10% by weight, based on the weight of the mixture comprising the calcium phosphate-based ceramics.

9. The method of claim 2, wherein the double distilled water is present at a content of 10 to 60% by weight, based on the weight of the mixture comprising the calcium phosphate-based ceramics.

10. The method of claim 1, wherein the calcium phosphate-based ceramics comprise any one selected from the group consisting of monocalcium phosphate monohydrate, monocalcium phosphate anhydrous, calcium metaphosphate, dicalcium phosphate dihydrate, dicalcium phosphate anhydrous, calcium pyrophosphate, octacalcium phosphate, α-tricalcium phosphate, β-tricalcium phosphate, calcium deficient hydroxyapatite, hydroxyapatite, tetracalcium phosphate and amorphous calcium phosphate, or a combination thereof.

11. The method of claim 1, wherein the preparing of the molded article formed of the ceramic paste comprises:
injecting the ceramic paste into a syringe to which an extruding machine is connected and then applying a pressure to the extruding machine based on the 3D rapid prototyping method to prepare the molded article.

12. The method of claim 1, wherein the drying of the molded article is performed at 25 to 60° C. for 12 to 48 hours.

13. The method of claim 1, wherein the sintering of the dried molded article comprises:
heating the molded article to a temperature of 1,100 to 1,200° C. at a rate of 1 to 10° C./min. and then cooling the molded article in a furnace while maintaining the molded article for 1 to 5 hours.

14. The method of claim 1, after the drying of the molded article, further comprising degreasing an organic binder included in the dried molded article by heating the molded article to a temperature of 500 to 600° C. at a rate of 0.1 to 5° C./min. and then maintaining the molded article for 1 to 3 hours.
